(19) United States
(12) Patent Application Publication
(10) Pub. No.: US 2018/0174669 A1
(43) Pub. Date: Jun. 21, 2018

(54) METHOD, COMPUTER PROGRAM PRODUCT, AND SYSTEM FOR EXCHANGING HEALTH DATA

(71) Applicant: OLEKSIY LAZARYEV, KAUNAS (LT)

(72) Inventors: OLEKSIY LAZARYEV, KAUNAS (LT); PLATON LAZARYEV, KIEV (UA); MARYAN KARPYK, POING (DE)

(73) Assignee: OLEKSIY LAZARYEV, KAUNAS (LT)

(21) Appl. No.: 15/736,779

(22) PCT Filed: Jun. 8, 2016
(86) PCT No.: PCT/EP2016/000939

§ 371 Date: Dec. 15, 2017

(30) Foreign Application Priority Data
Jun. 16, 2015 (EP) EP15001768

(51) Int. Cl. G16H 10/20; G16H 10/60; G16H 80/00

(52) U.S. Cl. CPC G16H 10/20; G16H 80/00; G16H 10/60

(57) ABSTRACT

A method for exchanging health data between a computer and a user includes providing a user database with the user's contact details and health data. The user database allocates the user to a user class based on the health data. A health question and a health recommendation are selected from a medical database for the user. The health question is transmitted to the user together with a multiple choice answer. An answer selected by the user is received by the computer. A user's state of health is stored in the user database based on the health data received in the form of the answer. A health recommendation is transmitted to the user. After a specific time interval, a query is transmitted whether the health recommendation was implemented. The user's answer on implementation is received. A user's conduct regarding the implementation of the health recommendation is saved in the user database.

### CROSS REFERENCE TO PRIOR APPLICATIONS

This application is a U.S. National Phase application under 35 U.S.C. § 371 of International Application No. PCT/EP2016/000939, filed on Jun. 8, 2016 and which claims benefit to European Patent Application No. 15001768, filed on Jun. 16, 2015. The International Application was published in German on Dec. 22, 2016 as WO 2016/202437 A1 under PCT Article 21(2).

### FIELD

The present invention relates to a method of exchanging health data between a computer system and a user, as well as to a computer program product with a program code for executing the method. The present invention also relates to a system for exchanging health data between a server and a user.

### BACKGROUND

Various information technology (IT) applications in medicine and medical engineering are already known from the prior art. Extensive data material from surveys is, for example, stored, managed and analyzed in special databases, in medical research (for example, as described in EP 1 521 200 A2). In medical engineering, medical devices are increasingly computer-controlled and integrated into computer systems (for example, as described in EP 1 883 197 B1). The introduction of the electronic health insurance card was most certainly a clearly perceptible change for a patient or a person with health insurance. Known IT applications nonetheless still exist in relative isolation from one another. In particular the patient or user is still not satisfactorily integrated into the existing systems and procedures.

An aspect of the present invention is to provide an improved method, as well as a system, for exchanging health data between a computer system and a user.

In an embodiment, the present invention provides a method for exchanging health data between a computer system and a user. The method includes providing a user database comprising contact details and health data of the user, the user database being structured so that the user is allocated to a user class based on the health data. A medical database comprising entries relating to user class-specific health questions, a number of predefined multiple choice answers for each of the user class-specific health questions, and user class-specific health recommendations is provided. A first communications interface in the computer system is provided for the user of the computer system. A user class-specific health question is selected, via the computer system, from the medical database for the user. A user class-specific health recommendation is selected, via the computer system, from the medical database for the user. The user class-specific health question selected from the computer system is transmitted to the user together with the predetermined multiple choice answer related thereto, wherein the computer system establishes a point in time when the user class-specific health question is to be transmitted based on details provided by the user in regard to the user's daily schedule and customs. An answer selected by the user is received by the computer system. A state of health of the user is stored in the user database based on the health data received in the form of the answer to the user class-specific health question. A user class-specific health recommendation selected by the computer system is transmitted from the computer system to the user. Once a predetermined recommendation time interval has lapsed, a query is transmitted to the user concerning whether the user class-specific health recommendation sent to the user from the computer system has been implemented. An answer of the user regarding whether the user has implemented the user class-specific health recommendation is received. A conduct of the user in regard to whether the user has implemented the user class-specific health recommendation is saved in the user database.

### BRIEF DESCRIPTION OF THE DRAWINGS

The present invention is described in greater detail below on the basis of embodiments and of the drawings in which:

FIG. 1 shows a flow diagram of the method in accordance with the present invention for exchanging health data;

FIG. 2 shows, by way of example, and as a schematic representation, the structure of a user database in accordance with the present invention;

FIG. 3 shows, schematically, a sample structure of a medical database containing general and user class-specific health questions;

FIG. 4 shows, by way of example, an excerpt from the medical database containing general and user class-specific health recommendations;

FIG. 5 shows the organization of a server according to the system in accordance with the present invention for exchanging health data between the server and a user; and

FIG. 6 shows a system for exchanging health data between a server and a user, as well as sharing said data with further healthcare protagonists.

### DETAILED DESCRIPTION

The method and system for exchanging health data or health information between a user and a computer system or server and any other parties participating in the data exchange, designed in accordance with the present invention, work according to a special communication protocol and have various advantages.

The present invention permits improved data exchange between various different parties in the public health sector which explicitly includes the person actually affected, namely, the user or patient. Improved data exchange provides that a doctor or medical care center providing treatment actually has all the medical information about a patient to hand, which provides more efficient treatment, combatting of illnesses, and more successful treatment. A comprehensive exchange of information between all the doctors providing treatment is important in especially critical situations of illness, which affects the healing of a patient, prevents complications, encourages improved health or, in certain circumstances, avoids the death of a patient.

The present invention also allows for improved documentation and gathering of health data. This may even be without any gaps so that, with recourse to such data, a doctor can select better therapeutic approaches for a patient and provide more successful treatment.

Finally, the present invention already provides an option for a patient or user to monitor his health values personally, or to control them himself/herself. Should there be problems or any impairment in the values, a doctor can be visited earlier and in a more targeted manner. Overall, the present invention will sensitize the user to his/her own state of health. A corresponding sensitization leads to the consequence of a lifestyle which is more focused on staying healthy so that the state of health of a person using the system can be sustainably improved. A patient who is informed about his/her current state of health and implements health recommendations can obtain motivating feedback via the method or system designed in accordance with the present invention. A reward system (scoring) can be implemented within the scope of such feedback which may provide a patient or user of the invention with genuine benefits, such as discounts on the premiums of insurance, etc.

The present invention also simplifies comprehensive statical analyses of both a medical and a business nature, which may contribute towards constantly continuing to improve the healthcare industry overall.

In an embodiment, the present invention provides a method for exchanging health data between a computer system and a user which includes the following steps:
providing a user database, wherein the user database contains contact details and health data of the user, and wherein the database is structured so that, based on the health data, a user can be classified as belonging to a particular user class;
providing a medical database, wherein the medical database contains entries concerning user class-specific health questions, a number of predefined multiple choice answers for each user class-specific health question, and user class-specific health recommendations;
providing a communications interface in the computer system for users of the system;
the system selecting a user class-specific health question from the medical database for a selected user;
the system selecting a user class-specific health recommendation from the medical database for a selected user;
transmitting the selected health question from the computer system to the user together with the associated plurality of answers available to choose from;
the computer system receiving an answer selected by the user;
storing the user's state of health in the user database based on the health data received in the form of answers to the health questions;
transmitting the selected health recommendation from the computer system to the user;
transmitting a query to the user, once a predetermined time interval has lapsed, concerning whether the health recommendation sent to him/her has been implemented by him/her;
receiving the user's answer about whether he/she has implemented the health recommendation;
saving the user's conduct in regard to whether he/she has implemented the health recommendation in the user database.

The computer system in accordance with the present invention may consist of one or more system components. The computer system can, for example, be a server or a system, the core of which is a server. A server offers the best infrastructure for communicating with a user or any other communication partner or enabling the exchange of data via corresponding communication interfaces.

According to the definition used in this patent application, a user is a natural person who is able to exchange health information or data with the computer system via a communication interface. The user may be a patient, but it is not necessary for the user to actually be ill. To that extent, the neutral term “user” is preferred over the term “patient” in the context of this patent application. This patent application also primarily concerns a special type of communication or communication technology, and not, for instance, a medical procedure in the narrower sense of the word. The user can, in turn communicate with the computer system via a corresponding communication interface. This may, for example, be done via a piece of software which runs on a computer, laptop, tablet, or even a smartphone. The user can, for example, make use of a portable device for the purposes of communicating with the system, as in this way it is possible for the computer system to trigger an interaction with the user nearly at any time, anywhere. The fact that, according to the present invention, the communication emanates from the computer system, and not the user, is important. Upon initially registering, a user no longer needs to get proactive himself/herself, but will, on his/her part, automatically be contacted by the system. A user can therefore not accidentally or inadvertently forget or neglect his/her health during the normal course of a day, but will be reminded about his/her health or healthy living in a very targeted manner, and even actively assisted in healthy living.

According to the present invention, a user database is provided, wherein the user database contains contact details and health data of the user, and wherein the database is structured so that, based on the health data, a user can be classified as belonging to at least one particular user class. The user's health data can in the process, for example, be entered into the user database with a greater or lesser degree of detail. A user can therefore in practice choose how comprehensively he/she would like to inform the system or personally use it. An entirely healthy person can enter, or arrange to have entered, in the user database health data such as his/her most important key data, such as age, weight, height, sex, etc. It is, however, also possible, and beneficial for a user, to enter his/her complete health data into the database, or arrange to have it entered, in co-operation with his/her doctor, in particular a complete case history. The architecture of the user database has been designed accordingly for this purpose. Based on the health data, the respective user is then allocated to at least one particular user class. This allocation can be performed automatically by the computer system. It may, however, also be laid down manually, for example, in collaboration with a doctor. It is thereby possible for the user classes to be designed consecutively or alternatively hierarchically, with main and sub-classes. Such a main class or generic class may, for example, be cardiovascular diseases, the particular attributes of which are, in turn, classified using sub-classes. The more fine-tuned the definition of a user class is, the more precisely the method and system in accordance with the present invention can be used to the benefit of the user. Between 1 and 20 sub-classes are, for example, typically stipulated for a basic illness, however, even more sub-classes may be stipulated.

The present invention also provides a medical database, wherein the medical database includes entries concerning user class-specific health questions, a plurality of predefined multiple choice answers for each user class-specific health question, and user class-specific health recommendations. The entries in the medical database can thus quite generally be divided into, firstly, health questions, into associated, predefined multiple choice answers, and health recommendations. The user class-specific health questions and the user class-specific health recommendations are, by their very nature, suitable for one or more user classes, and will also only be sent to a user in connection with the specifically admissible user classes. A major strength of the method in accordance with the present invention is that, besides the health questions posed, predefined multiple choice answers are already included in the medical database. The question and the associated multiple choice answers form a connected question/answer data set. This makes it easier for a user to answer the question. The answer is clear and unmistakable. That in turn facilitates a corresponding individual, or also statistical evaluation of the answers received. This makes answers of different users easier to compare or statistically more meaningful in the context of a statical analysis. The number of predefined multiple choice answers to choose from with a given question may vary depending upon the question. It is not necessary for the same number of predefined multiple choice answers to always be used for question/answer data sets. It may, however, be the case that the number of predefined multiple choice answers is the same for all or particular question/answer data sets. In that regard, not only Yes/No answers can, for example, be possible, but multiple choice answers can also be devised in a more sophisticated way. Three to five different multiple choice answers, or alternatively even more multiple choice answers, to a given health question can typically be saved as a data set in the medical database.

Any health questions associated multiple choice answers and/or health recommendations concerning all users, or that can be posed to all users, can also be saved in the database. Such questions, answers and recommendations are thus allocated to all user classes, and are denoted as general health questions, answers or recommendations. Within the scope of the present invention, however, at least one data set on questions with multiple choice answers and at least one data set concerning recommendations which are genuinely user class-specific in the narrower sense, and can thus not be used in general for all users, is included in the medical database. Having questions and recommendations that are specific to a particular user-class is a genuine strength of the present invention. Between 10% and 90% of all question/answer data sets and/or between 10% and 90% of all recommendation data sets are typically specific to a particular user class in the narrower sense. For example, at least 50%, for example, at least 90% of all question/answer data sets and/or at least 50%, for example, at least 90%, of all recommendation data sets are specific to a particular user class in the narrower sense. Within the scope of this patent application, the terms “health” or “health questions” and “health recommendations” can, for example, be broadly construed. In other words, all questions and recommendations that make meaningful reference to the health are also considered a health question or health recommendation. The term “health” thus not only covers the narrower field of medicine, but also the fields of sport, nutrition, lifestyle, etc., including fields which could have a significant influence upon the health of people.

According to the method of the present invention, a communications interface is provided in the computer system for users of the system. The system can, for example, via such a communications interface, communicate with a number of users at once. It is in particular also possible for a user to be permanently connected to the computer system via the communications interface, i.e., for the user to remain logged into the system. This considerably facilitates the communication with the user triggered by the computer system. It is also the case that, in social networks, for example, many users of such networks are constantly logged into said networks anyway, for example, via their mobile phones or smartphones.

According to the present invention, a user class-specific health question is selected from the medical database for a selected user by the system. A health question tailored to a particular user is thus quite individually automatically selected for the user by the system. The link making such a selection possible is the user's health data stored in the user database, or the allocation of the user to a particular user class, derived from the user's health data. It is, in this context, possible, for example, for the system to select the same health question from the medical database each time, for a user of a particular user class, when communicating with the user for the first time in a given day. It is, however, also possible for particular health questions that come into question for a particular user class to be selected in accordance with a particular algorithm or via a random sample procedure. Whatever health question is selected for a user of a particular user class, the health question fits the respective user and assists him/her in maintaining or restoring his/her health. When the process is taken further, it is also possible for answers given to the health questions by the user to influence the selection of a further health question by the system, and for the selection to thus be based on a user's answer. Corresponding algorithms can be implemented in the method in accordance with the present invention.

The present invention similarly works with the procedural step of the system selecting a user class-specific health recommendation from the medical database for the selected user. Also in this case, the user in any case receives a selection adapted to his/her user class, which is made by the system automatically. In this context, health answers already given by a user may also be taken into account. The health recommendation can, for example, concerns a special recommendation for action which a user can execute in the short term, for example, on the same day or within the next few hours.

As a further procedural step, the health question selected is sent to the user by the computer system together with the associated number of predetermined multiple choice answers. It is, for example, in practice the case that the corresponding health question is displayed on a data terminal used by the user, together with the multiple choice answers, in a visually appealing format. The selected health question is, again, sent by the computer system automatically. It may be sent at a time selected by the system, however, it is also possible for the selected health questions to be sent when a user re-connects with the computer system or logs into the system. It is, however, also more expedient here for the user to be permanently logged into the system in order to trigger the health question being sent to the user by the computer system at an ideal moment. According to an embodiment of the present invention, it is the case that the system can, for example, establishe the point in time for sending the selected health question based on details given by the user regarding his/her daily schedule and habits. These are, for instance, enquired about whenever a user newly registers. The system analyzes these details, and then determines an optimum point in time to send the question. That may, for example, involve the health question being sent at the same time each day, it may, however, also be the case that the questions are sent on different days of the week at different times. The method in accordance with the present invention can thereby also assist a user of the system in a time-wise optimized way.

In a next step, an answer selected by the user is received by the computer system. That means that the user has selected an answer that fits best for him/her from the multiple choice answers suggested. The user can, for example, send the answer selected by him/her to the computer system promptly after receiving the health question. This is an indication that the user is committed to using the system. It is also possible to define a particular time interval within which the user's answer needs to have been received by the computer system. The answer will otherwise be deemed not to have been given. This is especially important if the user's usage patterns are being analyzed or if the user wishes to draw further benefits for himself/herself from using the system if a scoring or reward system is in place.

The state of health is afterwards stored in the user database based on the health data received in the form of an answer. In this context, it is possible for the data itself to be saved in the user database, which then contains exact details on the user's state of health, for example his/her blood pressure, insulin level, etc. It is, however, also possible for an analysis or evaluation of the health data to be performed prior to actually saving it, and only general details to be stored in the user database, such as “Values OK”, or, conversely, “Values not OK”.

A further procedural step involves the selected health recommendation being sent to the user by the computer system. The selected health recommendation may be sent to the user immediately after an answer to a health question is received from the user. It is also possible, however, for both the health question, along with the predetermined multiple choice answers, and the health recommendation to be sent to the user simultaneously. It is naturally also possible to first send the health recommendation and to only send the user the health question at a later point in time. The sequence of the individual procedural steps of the method in accordance with the present invention is to this extent not strictly prescribed, or not strictly coupled with the sequence of the individual procedural steps set forth in the appended claims.

In a further procedural step, once a predetermined time interval has lapsed, a query is sent to the user concerning whether the health recommendation sent to him/her has been implemented by him/her. It is important to define such a time interval in regard to the recommendation because it is natural that the user needs some time to implement a health recommendation. The purpose of the health recommendation is not primarily to enquire what the user has already done for his/her health, but to assist him/her in actively implementing steps to maintain his/her health in the course of his/her day. In the simplest case, the enquiry sent to the user only requires a “Yes” or “No” answer. It is also possible that, if the answer is in the affirmative, a more in-depth follow-up question is transmitted to the user by the system which is aimed at determining more precisely the degree to which the health recommendation has been implemented. A health recommendation could, for example, read as follows: Go for a walk for a few minutes today! In response to the enquiry, “Did you go for a walk today?” a follow-up question could then be transmitted to the user by the system concerning how many minutes the walk lasted or what distance was covered. This could, in turn, be performed in the form of a multiple choice question with predetermined multiple choice answers.

After receiving the user's answer regarding the implementation of the health recommendation, the usage pattern in regard to implementing the health recommendation is saved in the user database. The wording of the health recommendation itself and the implementation by the user can, for example, be stored in the user database in the form of a positive or negative entry. It is also possible to only store the number of health recommendations implemented in the user database. It is possible to set the number of health recommendations implemented in relation to the number of health recommendations that have not been implemented. The usage pattern in regard to implementing a health recommendation can, for example, be stored in the user database in minute detail.

In an embodiment of the present invention, the user can, for example, be evaluated in regard to his/her usage pattern concerning the system, and in particular in regard to executing the health recommendations or recommendations regarding action to be taken. Such an evaluation may be undertaken each time that the system has interacted with a user or at regular intervals, for example, once per day or once per week, or also upon the user requesting the system to do so or at the request of a third party. This evaluation step is not primarily intended to ascertain whether the user's state of health is good or poor. The aim is, rather, to objectively assess whether the user of a system is actively participating in the procedure, i.e., answering health questions and following health recommendations. Should the user act actively and positively for the benefit of his/her health, a positive evaluation is given. The evaluation itself can, in that respect, be carried out by a point system (scoring). It is, for example, possible to award a particular number of points for each health recommendation complied with. The associated number of points can, for example, be stored in the medical database. It is possible for various different health recommendations or the implementation thereof to be awarded different numbers of points. There are therefore, as it were, various levels of difficulty in complying with health recommendations. Active and health-conscious behavior is thus evaluated positively as part of the method in accordance with the present invention.

It is possible, as a further procedural step, for a user to be informed about his/her current assessment. A corresponding message may, for example, be sent to him/her. It is also possible for no message to be sent automatically, but rather, for the user to be able to check what score is currently stored for him/her in the user database at any given time by initiating a corresponding enquiry. A positive evaluation will prompt a user to continue using the system, especially if other tangible economic benefits can be linked to it for him/her. It is, for example, conceivable for a health insurer to offer cheaper premiums to a user who actively takes precautions to preserve his/her health, or basically follows principles of healthy eating or engages in conduct conducive to good health, via corresponding contracts.

In an embodiment of the present invention, a user class can, for example, be defined based on at least one of the following criteria: age of the user, sex of the user, illness, degree of severity of an illness, complications associated with an illness, medication. Lifestyle may also serve to define a user class. The user class defined based on such a criterion may define an independent user class, a main user class or a sub-user class. It is, for example, possible, to define a main user class via a particular illness. Further sub-classes may then define the degree of severity of the corresponding illness, a complication with such an illness, and the corresponding medication. In this example, there is, then, a main class and there are also three sub-classes. A more refined or entirely different sub-division of user classes is, naturally, also possible. In an embodiment, a user class can, for example, be defined by a combination of a main class and at least one sub-class. Such a user class definition can, for example, follow a hierarchical or tree structure. The user class definition is overall to be made dependent upon with what degree of complexity or detail the method in accordance with the present invention or computer system is supposed to operate.

In an embodiment of the present invention, the method can, for example, also include the following step: regularly checking the allocation of a user to a user class and, if necessary, updating his/her user class accordingly. It may, in addition, also be the case that, following initial registration, the user is accorded an initial classification. This initial classification may then be subsequently reviewed, and, if necessary, refined. That means, for example, that the classification starts with a main class which is then, for instance, supplemented by one or more sub-classes in the course of using the procedure. Classes can, naturally, also be entirely exchanged or updated if necessary. The method in accordance with the present invention can thus also be extended in regard to allocating a user to a user class, and is capable of learning.

In an embodiment of the present invention, at least one health question in the medical database can, for example, be allocated a repetition interval as a parameter, which specifies at what intervals the same health question is re-sent to the same user. This can be the case with all health questions. There are, for example, health questions that the same user is repeatedly asked every day, every second day or every week, etc. That means that a health question recurs at particular time intervals. Various different health questions may be accorded different repetition intervals. The repetition interval may therefore expediently be defined for the respective health question on a quite individual basis.

In an embodiment of the present invention, the parameters stored in the medical database can, for example, include both objective and subjective health parameters of a user. The health parameters are, in that respect, in particular determined via the possible answers to a particular health question. These multiple choice answers belong to a particular type, and may be sub-divided into objective and subjective health parameters. All multiple choice answers associated with a particular health question can, for example, belong to the same parameter type. An objective health parameter is understood to be a measurable health parameter. The latter includes, for example, the blood pressure, the pulse, the body weight, the body fat percentage, the blood sugar, the insulin level, etc. But subjective health parameters of a user can, for example, also form part of the health data stored in the medical database. In response to the health question, “How did you sleep last night?”, a user may, in principle, only give a subjective answer which best reflects how he/she perceives the situation. He/she may, for example, answer that he/she slept very badly, badly, well or very well. From an objective perspective, only a particular duration of sleep would have been able to be established. Such an objective parameter does not, however, adequately reflect a user's subjective perception regarding the topic of sleep. For this reason, in order to maintain a comprehensive health profile, the subjective parameters of a user can, for example, also included among the health parameters so that at least one corresponding data set of this subjective type exists. The implementation of subjective health parameters in the method in accordance with the present invention also has the positive effect that a user perceives the health questions as being more attuned to his/her person, and considers them a more personal approach. It is precisely not only abstract values that are concerned, but his/her very personal state of health. If this aspect of a user is addressed, this provides that the user co-operates better with the method in accordance with the present invention, and the method is able to give better and more positive support in regard to promoting a user's health.

In an embodiment of the method of the present invention, a health question and/or health recommendation can, for example, be selected by the system based on a feedback loop. In the process, when making such selection, data received by the system in the form of user answers to the health questions and/or in the form of user details on the implementation of health recommendations is resorted to. This makes it possible for the method to be customized even more to the respective user and his/her needs.

In an embodiment of the present invention, the method in accordance with the present invention furthermore involves the following procedural step:
Providing a further communication interface for a further protagonist from the healthcare sector, in particular for a doctor, a hospital, a pharmacy, a health insurance company.

The above list is not conclusive. It does, however, make clear that the method in accordance with the present invention is able to very comprehensively, in the best case, integrate all the healthcare sector protagonists into the procedure. It is, in this context, naturally not the case that each healthcare sector protagonist has full access to the user database or the medical database. It is instead designed so that certain data can accordingly be accessed depending upon the type or class of the protagonist. It is thus possible for a protagonist to actively, i.e., also inherently, read a user's entry in a database of the computer system via this further communication interface, in line with his/her access rights. In addition, or alternatively, it is even possible for write access to be granted to the system. It may be the case, for example, that after prescribing a particular medication in consultation with his/her patient or user of the method in accordance with the present invention, a doctor enters the medication prescribed into the user database, which is included with the user's health data by the database. The method in accordance with the present invention can therefore be extended from many perspectives. It is additionally or alternatively possible for the computer system to only communicate with the further protagonist outwardly in one direction. Under this aspect, the targeted passing on of information/data to a further participant or protagonist of the system (in consultation with and with the authority of the user) is concerned.

In an embodiment of the present invention, the health data in regard to a user can, for example, automatically be exchanged between the computer system and the further healthcare sector protagonist. This of course occurs in practice, while taking into account data privacy provisions and data access rights of the further protagonist in the computer system. Health data concerning a user can, for example, be exchanged between the computer system and a number of healthcare protagonists simultaneously and, in particular, in real time. That means that the case of health data concerning the user stored in the user database simultaneously being notified to the user personally and a healthcare protagonist, such as the user's doctor, is also covered. It is thereby possible to keep various different healthcare protagonists up to date in regard to the health of a particular user simultaneously. The simultaneous exchange of health data may, in that respect, include health data in the narrower sense, but also the evaluation of the user's usage pattern in regard to the system. It is, for example, possible for a doctor, a hospital, a pharmacy, or a health insurance company to be simultaneously informed by the computer system about the user's current score in regard to the user's usage pattern so that the user can redeem a bonus coupon corresponding to the score when next visiting the pharmacy, the doctor, the hospital, or the health insurance company. In practice, a corresponding entry is, for example, made in the user database concerning which protagonist, if any, is also integrated into the overall procedure or system in regard to the user concerned, and at what level.

In an embodiment of the present invention, the method in accordance with the present invention can, for example, also include the following step:
Analyzing the efficacy and/or the cost-effectiveness of health-promoting measures.

Data from the user database and/or the medical database can, in the process, be examined using basically well-known statistical methods. It can, for example, be ascertained whether a particular medication to counteract a particular illness is marked by an above-average success rate, or, alternatively, whether it has more side effects than average. The influence of the healthy lifestyle on the occurrence or severity of illnesses, which can otherwise only be ascertained with difficulty, can be investigated better based on the method in accordance with the present invention. These so-called soft parameters are practically not accessible using traditional methods, and can least of all be evaluated in a statistically meaningful or significant way.

The method in accordance with the present invention described above and its embodiments can be combined with one another, in whole or in part, as long as no technical conflicts arise as a result. It is in particular possible for methods in accordance with the present invention to be carried out a number of times, in whole or in part. The competent specialist will recognize which combinations are technically feasible and expedient. The sequence of the individual procedural steps may vary, in the event of individual procedural steps not being dependent upon one another or building upon one another.

A further aspect of the present invention relates to a computer program product with a program code for executing the procedure, as described above, either in general terms or in regard to particular embodiments. The computer program product can, in that respect, exist in either corporeal or non-corporeal form. The program code does not need to be written in a particular programming language. Only a programming language is supposed to be used that is appropriate to the problem. The essence of the present invention in the computer program product does not lie in the program code itself, but in the technical method underlying the program code, as described above. The computer program product can in particular be designed in one piece or in a number of parts, for example, with a program code that is sub-divided into modules.

The programming languages for programming the method may, for example, consist of a combination of standard programming languages and standards, such as HTML5, Java, JavaScript. That allows for executing the method irrespective of the respective operating system (Windows, Linux and similar, Android, iOS, etc.). The list of programming languages is not laid down and will also change as computer technology advances.

A further aspect of the present invention relates to a system for exchanging health data between a server and a user which includes the following:
a user database with user information in regard to the user's contact details and health data, wherein the database is structured so that that user is allocated at least one user class, based on the health data;
a medical database with user class-specific health questions and a number of possible associated answers in each case, as well as with user class-specific health recommendations;
a program code for communication management between the server and the user;
a program code for automatic healthcare support, which is programmed to automatically select a user class-specific health question with associated possible multiple choice answers to be sent to the user, and which is also set up to select a user class-specific health recommendation for the user, to be sent to the user;
a program code for automatically recording the user's state of health based on health data in the form of an answer transmitted to the server by the user; and
a program code for automatic control over the execution of health recommendations received based on details transmitted to the server by the user.

The system for exchanging health data in accordance with the present invention is in particular suited to execute the method for exchanging health data between a computer system and a user in accordance with the present invention as described above. The program codes mentioned above are set up or programmed in exactly such a way that the corresponding procedural steps can be processed by the computer system or the server automatically. Some or all of the databases, and some or all of the program codes, can, for example, be stored on the server, in other words the server contains the databases and program codes. What has already been stated above about the method in accordance with the present invention also applies to the system in accordance with the present invention. Explanations concerning the terminology in connection with the method apply equally in connection with the system in accordance with the present invention.

Attention is additionally drawn to the following:

The program code for communication management between the server and the user allows for providing a communications interface in the computer system for users of the system, as well as, if applicable, also for any other healthcare protagonists. The transmission and receipt of data can, for example, also be handled via this program code.

The program code for automatic healthcare assistance permits health questions, along with associated multiple choice answers, and health recommendations to be selected from the medical database.

The program code for automatically recording the state of health in particular allows for saving health data received from the user in answer form. It is, for example, also possible to store new health data received in the user database by other means.

The program code for automatic control over the execution of health recommendations received allows for storing the user's behavior in regard to the health recommendations in the user database.

In an embodiment of the present invention, the server can, for example, have a program code for automatically evaluating the user in regard to his/her usage pattern in regard to the system, and in particular in regard to his/her execution of the health recommendations. A scoring for the respective user can thus, for example, be created and/or administered with the aid of the program code.

In an embodiment of the system, a user class can, for example, be defined based on at least one of the following criteria: age of the user, sex of the user, illness, degree of severity of an illness, complications associated with an illness, medication. The lifestyle of users may also be drawn upon to define a user class.

In an embodiment of the system, a user class can, for example, be defined by a combination of a main class and at least one sub-class.

In an embodiment of the system, a program code for regularly checking the allocation of a user to a user class and, if necessary, updating his/her user class accordingly can, for example, be provided for.

In an embodiment of the system, at least one health question in the medical database can, for example, be allocated a time interval for repetition as a parameter, which specifies at what intervals the same health question is re-sent to the same user; this can also be the case with all health questions.

In an embodiment of the system, the health data received by the server in answer form can, for example, include both objective and subjective health parameters of a user.

In an embodiment of the system, at least one health parameter from the following list can, for example, be used: blood pressure, pulse, body weight, body fat percentage, blood sugar.

In an embodiment of the system, the system can, for example, include a communications interface for at least one further healthcare sector protagonist, in particular a communications interface for a doctor, a hospital, a pharmacy or a health insurance company.

In an embodiment of the system, the server can, for example, furthermore have a program code for automatic communication between the server and the further healthcare sector protagonist, wherein health data concerning a user is exchanged between the server and the further healthcare sector protagonist within the context of the communication.

In an embodiment of the system, health information concerning a user of the system can, for example, be exchanged between the server and a number of the healthcare sector protagonists at once, and in particular in real time.

In an embodiment of the system in accordance with the present invention, the server can, for example, also comprise a statistics program code which is programmed to analyze the efficacy and/or economic feasibility of health-promoting measures.

In an embodiment of the present invention, the system can, for example, include a mobile user terminal, in particular a smartphone or tablet, via which the user communicates with the system. The mobile user terminal has the advantage that it is possible to communicate with the user at virtually any location and at any time in just exactly the way that best promotes a user's state of health.

It likewise applies to the system in accordance with the present invention that one or more variant embodiments of the system can be combined with one another, in whole or in part, as long as no technical conflicts arise as a result.

The present invention can be better understood under reference to the drawings.

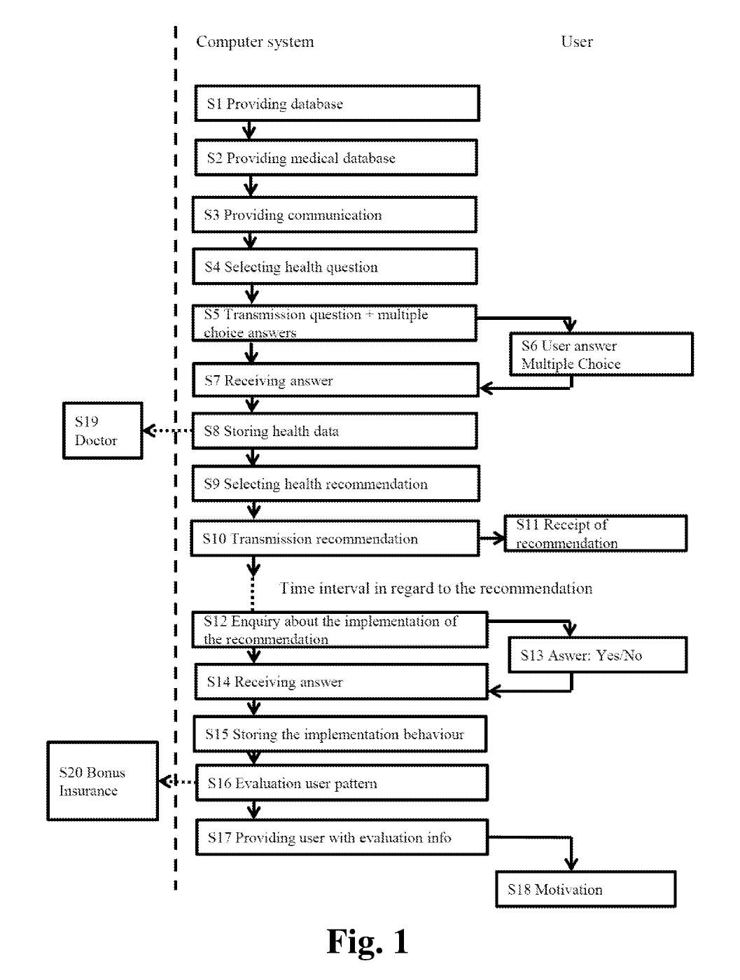

FIG. 1 illustrates, in the form of a flow diagram, the method in accordance with the present invention for exchanging health data between a computer system and a user. In that respect, the exchange of the health data is initiated by the computer system. That means that the action is always initiated by the computer system, and the user (after he/she has once logged into the system or registered with it) responds to questions posed by the computer system or the server. The procedural steps are therefore also implemented by the computer system; it is only necessary for the user to be able to enter data, or transmit data, to the computer system. The user thus only requires a technically very simple user interface which can, for example, be provided via a simple app for smartphones.

A user database is provided with the procedural step 51. User information concerning contact details of the user, and also health data of the user, is stored in this user database. The user database has a special structure. In fact, each user is allocated to a particular user class based on the health data. The further procedure involves checking the user class in order to select or lay down the respective action on the part of the system concerning health-promoting measures for a user.

A medical database is provided in procedural step S2. The medical database has a special structure and is one of the core elements of the present invention. Entries concerning health questions and health recommendations can be found in the medical database. Each health question is allocated a large number of multiple choice answers in the database. There is thus a number of possible answers stored in the database that a user can give in response to the health question (multiple choice). Every health question is also allocated to one or more user classes. It is also possible for a health question to be of a general nature, in other words, belong equally to all user classes. The health recommendations stored in the database are not questions, but health recommendations for a user that are supposed to encourage him/her to actively carry out the recommendation and, as a result, positively influence his/her health. The health recommendations stored in the database are also allocated to one or more user classes. General health recommendations are allocated to all user classes.

In procedural step S3, a communications interface is provided in the computer system for users of the system. That basically results in communication between the computer system and the user being enabled.

In procedural step 4, a health question is selected from the medical database by the system for a particular user. This procedural step is thus carried out automatically and is triggered by the system itself; it is neither stipulated nor necessary for the user to submit a corresponding enquiry to the system. The health question selected may be a general one, or a user class-specific health question in the narrower sense. In any case, it is specifically suited to the specially selected user who is concerned at that moment.

In the next procedural step, S5, the example shows the health question selected by the computer system being sent to the user together with the associated various pre-defined multiple choice answers to the health question selected. In step S6, the user who has received the question answers by selecting a multiple choice answer. In practice, the health question, along with the multiple choice answers, is displayed in the corresponding app on a mobile user terminal of the user, and the user selects the answer that is correct in his/her case, for example, by way of a touch screen input. This is then sent back to the system, or, in procedural step S7, received by the computer system. It is possible for the user to be asked to re-confirm the correctness of his/her data entry prior to sending it. It is, however, also possible for the answer to be sent to the system immediately after entering it.

After the answer has been received in procedural step S7, in procedural step S8, the user's state of health is stored in the user database, based on the health data received. In the embodiment described, the question and answer protocol of the communication between the system and the user is stored in the user database. It is also possible for only the data itself to be stored, or even for an evaluation of the data to be stored immediately, without the data itself (for example: “blood pressure in the normal range”, instead of the specific blood pressure). It is, however, easier, and more meaningful, to actually store the health values themselves, for example, in combination with the point in time of the value being entered on a user terminal or the time of receipt by the system.

In the method in accordance with FIG. 1, in a further procedural step S9, a health recommendation is automatically selected from the medical database by the system for the user concerned. It is possible in this context for the user's answer from procedural step S6 to have already been taken into consideration when making this selection. It is, however, also possible for the health recommendation to be automatically selected by the system independently of said answer. The selection can naturally also already be made at an earlier point in time in the procedure, for example, at once with or directly prior to or after procedural step S4.

The health recommendation selected in procedural step S9 will then be sent to the user in procedural step S10. This contains the health recommendation from procedural step S11. A pre-defined time interval in regard to the recommendation is linked to the user class-specific health recommendation stored in the database. This interval specifies what period of time should pass before the computer system actively enquires whether the recommendation received was implemented by the user. The length of this time interval in regard to the recommendation is in practice measured so that the user has sufficient time to implement the recommendation. In that respect, the time interval in regard to the recommendation is dependent upon the respective type of recommendation. Thus, an individual allocation of a time interval in regard to the recommendation to each individual health recommendation stored in the database is concerned.

In procedural step S12, once the time interval in regard to the recommendation has lapsed, the user is then sent a query about whether the health recommendation sent to him/her was implemented. In the simplest case, this query constitutes a question that is to be answered with “Yes” or “No”. In step S13, the user gives the corresponding answer. In step S14, the user's answer is received, and, in step S15, automatically stored by the system. In procedural steps S8 and S15, the health data stored about a user in the user database is consecutively supplemented. A meaningful data history emerges.

In the optional procedural step S16, the user is then evaluated in regard to his/her usage pattern in regard to the system, and in particular in regard to his/her execution of health recommendations. This step may, for example, be implemented by points being awarded for the user's behavior. For example, one point might be given each time a health question is answered, and a further point every time a health recommendation is implemented. A different number of points may also be awarded, however, for answering health questions and for implementing health recommendations, depending on the weighting or health benefit. What is important or advantageous at this point is that it is not the medical values of the user personally which are taken into account in the assessment of the user behavior, but that the user's co-operation in connection with the exchange of the health information is assessed. A user who answers the health questions on a very regular basis and pays attention to his/her health, by implementing the recommendations, for example, receives a more positive assessment than someone who is in fact of sound health but practically never uses the system. To that extent, points are awarded for health-promoting conduct, or conduct which maintains health, not for the health itself. The method is, to that extent, not discriminatory. The evaluation performed in step S16, or associated score, is then, in turn, stored in the user database.

It is possible, and also advantageous, for the assessment to be used in a further procedural step S17 to inform the user. The latter thereby, in step S18, is informed, and moreover possibly motivated. The motivation, in that respect, is firstly achieved through being made aware of the assessment itself, which is comparable to the user being praised. Secondly, the user may, when implementing the method in accordance with the present invention, accordingly also receive worthwhile financial rewards from a provider of the method. An example of that would be his health insurance premiums being reduced.

It is, for example, also possible for a user of the system to be able to inspect the complete data stored about him/her in the user database at any time. It should, however, be made impossible to manipulate the data. He/she can thus also personally become aware of his/her health data, and in fact of the complete data stored in the database. That may be the health data that has been stored in the health database in the course of executing the method in accordance with the invention. It may, however, further, also be the specialist data which he/she has had ascertained by his/her doctor. It is explicitly desired for doctors to also be involved in the procedure accordingly. If a user so wishes, it is, for example, possible, in the course of procedural step S19, to at once also transmit the health data currently obtained to the user's doctor. This may, in turn, be done fully automatically. It is alternatively possible for the user to only access his/her data via the doctor at the time of the next visit or for the data to only be provided to the doctor upon specifically being requested to do so. There are many possible ways to diversify the method in accordance with the present invention, add new and beneficial options to the procedure, and utilize all the benefits of the method in accordance with the present invention.

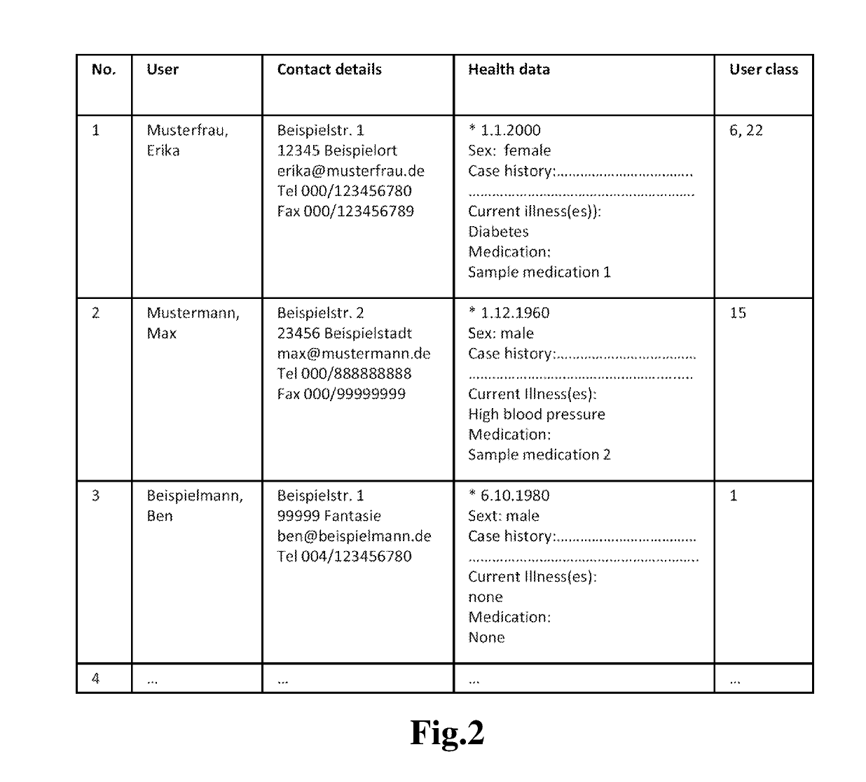

FIG. 2 shows, by way of example, and as a schematic representation, an excerpt from a user database. Every user is allocated an individual user number in the shown example. Firstly, the user's contact details and, secondly, his/her health data, are recorded in the user database. This involves at least one particular user class also being allocated to the user personally. The contact details include the usual contact details, such as address, e-mail address, telephone number, or fax number. The health data may include various pieces of information about the patient relating to health being stored. For example, the user's date of birth and sex are recorded. Current illnesses and any medication currently taken are stored with the health data in accordance with FIG. 2. A complex case history for the user can also be stored with the health data. Such a case history may be of any length and comprehensive, and is therefore only indicated by a row of dots in FIG. 2. It will be clear to the competent specialist how such a case history can be designed. It is very important that at least one user class is allocated to the respective user in the user database. Simple figures are given to express the user class in the shown example. It is, however, also possible for the user classes to have a tree structure so that main classes or generic user classes can be used in combination with ancillary classes or sub-classes. This makes it possible, for example, to differentiate, or to gain a better overview when linking the user classes to the health questions and health recommendations. It is, for example, the case that particular health questions should be posed to all users with heart/cardiovascular diseases. These would then be allocated to the same main user class. Depending upon the specific heart/cardiovascular disease, a user sub-class could then be defined, where health questions and health recommendations can, in turn, be differentiated according to the user sub-class. A further sub-class may, for example, define the degree of severity of an illness or the medication in the case of a particular illness. It is in this way possible to allocate at least one quite individual user class, relevant to him/her personally, to every user of the method in accordance with the present invention. This allows for providing the user with targeted health assistance. It is, in this context, also possible for an allocation of the user to a particular user class to be reviewed, or, if applicable, updated and/or refined, at regular intervals. That means that it is possible at any time to provide the user with optimum assistance based on his/her user class allocation.

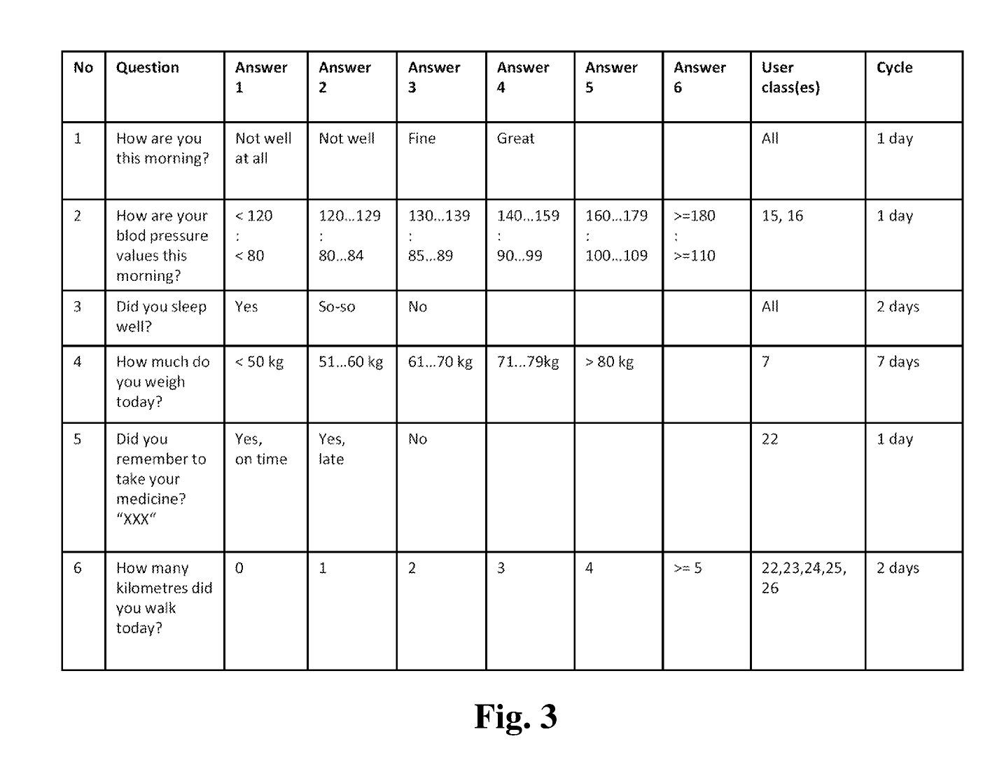

FIG. 3 shows, by way of example, and as a schematic representation, an excerpt from the medical database in accordance with the method in accordance with the present invention. The entries concerning health questions in the medical database are shown. Each question is allocated a key, or alternatively a particular code. Besides the question itself, several multiple choice answers for every question are recorded in the medical database. The number of multiple choice answers may vary from question to question. In the example shown, Question No. 2, “How are your blood pressure values this morning?”, has six possible multiple choice answers. The answers then relate to particular respective intervals of the systolic and diastolic values. These multiple choice answers thus concern objective medical data, i.e., data that has been precisely and objectively measured. An example of the opposite kind is Question No. 1 in FIG. 3, “How are you this morning?”. This is a subjective health question which any user can answer individually, according to how he/she feels at the time. The multiple choice answers 1 to 4 are “Not at all well”, “Not well”, “Fine” and “Great”.

All health questions, regardless of whether they are subjective or objective, are allocated to at least one user class. In most cases, a number of user classes are allocated to a given question. A particular cycle is also laid down for each health question in the medical database. This cycle specifies how often the question is posed to the same user, or how often the question is thus repeated. A particular measuring sequence with a particular measurement interval thereby emerges. In the example shown, the question cycle varies between one day and seven days. The cycle is individually adapted to the respective question. The question, “How much do you weigh this morning?” is allocated a seven-day cycle, as the tendency to lose or gain weight can only be determined with any significance after a number of days. Daily measurements are deceptive, and frequently cause the user entirely unnecessary stress.

It is also possible for a particular health question to still have open parameters or content parameters that are occupied by a user's data from the respective user database. In the example Question No. 5 in FIG. 3 is of this type. The question is: “Did you remember to take your XXX medicine?” In this case, XXX is a content parameter. Here the system can thus, when asking the question, link the individual medication of the user with health question No. 5 from the info on medication in the user database. The more individual a user of the method or system in accordance with the present invention finds a health question, the more positive his/her answering behavior in regard to the health questions will be. This in turn brings about a more positive effect towards a more health-conscious lifestyle and way of life.

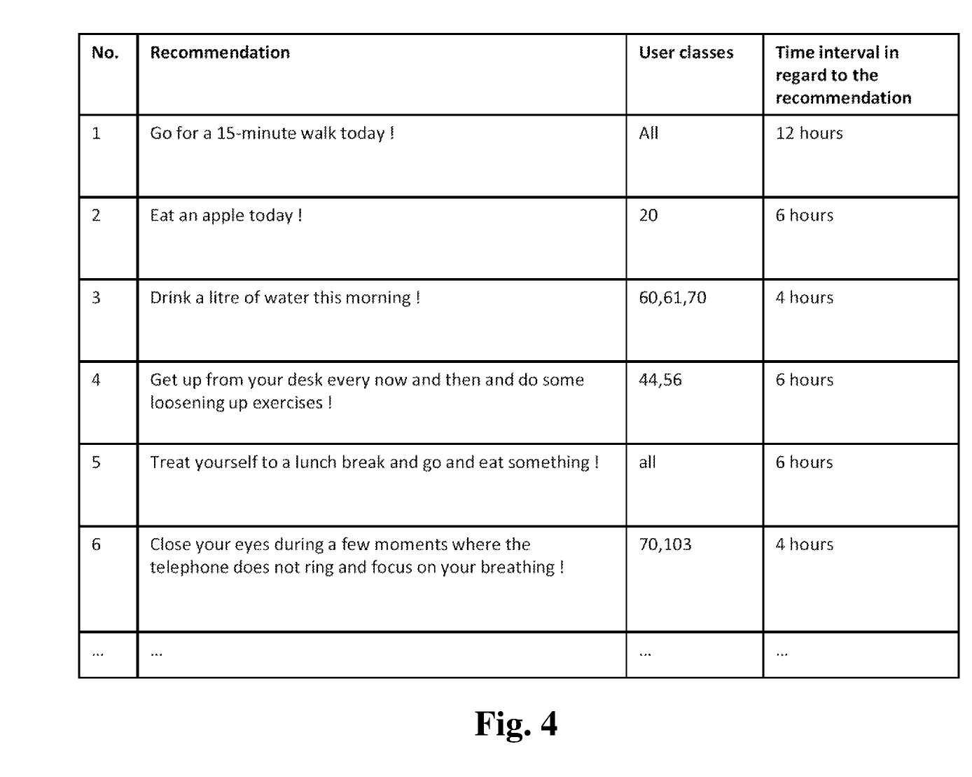

FIG. 4 shows a further excerpt from the medical database in accordance with the present invention, wherein the entries in regard to health recommendations are shown by way of example in this database excerpt. The health recommendations are, in turn, allocated an identification number, or alternatively a particular code. Every health recommendation is allocated to one or more user classes. There are thus general health recommendations (allocation to all user classes) and user class-specific health recommendations. A so-called time interval in regard to the recommendation is, moreover, stored in the database for each recommendation. This time interval in regard to the recommendation is implemented in the communication between the computer system and the user so that precisely this time interval lies between the sending of the recommendation and asking the user whether the recommendation was implemented. The time that is typically also appropriate to give a user the opportunity to implement the recommendation properly is also taken into account when determining the interval. In other words, the recommendation is not supposed to cause the user any stress. The query should instead be formulated as a “soft question”, as it were, or, as the case may be, as a reminder. The health recommendations are typically formulated by a health expert, and in particular put in relation to the user classes defined.

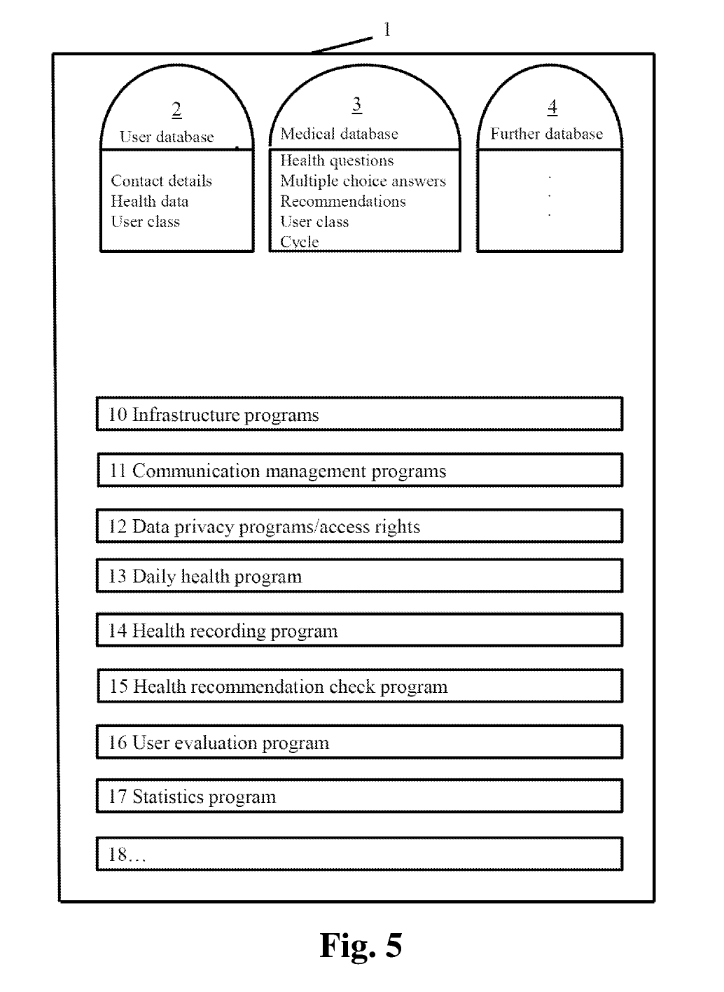

FIG. 5 illustrates, as a schematic representation, the configuration of a computer system 1 or server 1 which is suitable for executing the method in accordance with the present invention. The server 1 contains various databases 2, 3, 4, etc. Database 2 is the user database 2 which contains user information in regard to the user's contact details and health data and wherein the user is allocated to a particular user class based on the health data. The health questions with associated multiple choice options, as well as the health recommendations with their cycle, are stored in the medical database 3. Both health questions and health recommendations are allocated to one or more user classes.

The database 4 may contain further data which is expedient for the method or system. It is, for example, possible for the user class definition itself to be stored in a further database 4. It is especially expedient to define the user classes in a separate database if a system of both main user classes and one or more ancillary user classes is used. With such a tree structure, it is then possible to obtain a better overview of the user classes, and any additions or amendments required can be integrated into the system structure more easily.

Besides the databases 2, 3, 4, etc., the server contains various program codes. These may manifest in the form of separate programs, or in the form of various program components of a superordinate program. The latter in particular include infrastructural programs 10, for example, aimed at server management, cloud applications, storage and data processing, etc.

Communication management programs 11, which enable communication or an exchange of data between the server 1 and a third party, are also provided for in the shown example. The sending of health questions and health recommendations, as well as the receipt of the associated user answers, is in particular also handled via such communication management programs. The third party mentioned above may be a user of the system, or alternatively another healthcare sector protagonist, such as a doctor, a pharmacist, an insurance company, a hospital, etc.

In particular when there is a large number of different communication partners, it is necessary to store various data privacy programs and access rights on the server 1 in a special code 12. It is thereby provided that only authorized protagonists obtain access to particular data in the system. Such access rights may be uniform for various different protagonists, or alternatively be laid down so that they are individual to particular users, depending upon the user's consent.

What is furthermore stipulated for the actual course of the method in accordance with the present invention is a program code for automatic health assistance 13, in particular on a daily basis. With the aid of the program code, health questions, including the associated multiple choice answers, to be sent to the user automatically are selected. Within the same program code, the selection of health recommendations for a user can also be implemented. Such a program code will typically contain a number of components, at least one for selecting the health questions and a further program component for selecting the health recommendations. It is, however, in this context, also possible for the selection of recommendations for action to also be aligned with answers previously received from the user to a selected health question. There are also feedback facilities concerning user behavior or user answers within the program code.

A program 14 with a program code for automatically recording the state of health is also provided for. In the process, in particular the health data transmitted to the server by the user in answer form is stored in the user database 2, either directly or indirectly.

A program code 15 for automatic control over the execution of health recommendations received is likewise an important component of the computer system 1. This involves, for example, ascertaining and saving information about whether a user has implemented the health recommendations received—and, if so, how.

The computer system 1 optionally ultimately contains a program code 16 for automatically rating the user. That does not mean that the user's health values themselves are rated, but rather the user's usage pattern, and in particular whether, and to what extent, the user carries out health recommendations. Points may be awarded for good co-operation, for example, which may also make the user eligible to receive bonuses from a health insurance company, for instance.

In the example according to FIG. 5, a statistical program code 17 is, moreover, provided for, which is, for example, programmed to analyze the efficacy and/or economic feasibility of health-promoting measures. The statistical program may offer options for making general statements on the improvement in the state of health, with particular user groups. It is possible to investigate the effectiveness of particular medications, or side effects occurring with particular medications. It is possible to analyze overall user behavior or cooperation on the part of users within the context of the present invention and thereby further improve the implementation of the present invention, if applicable. There are many options for implementing a statical method which permit interesting conclusions to be drawn in regard to medical, sociological, or economic questions.

Further programs 18 can of course also be integrated into the computer system 1.

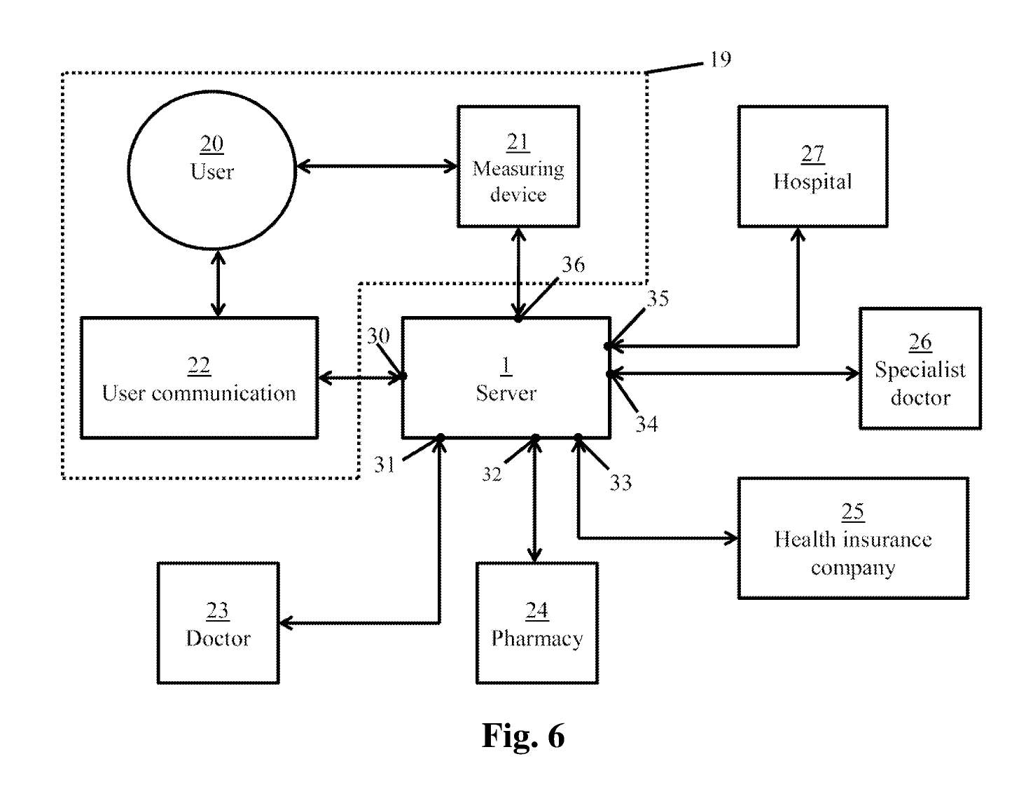

FIG. 6 shows once again by way of example the computer system or the server 1 and its cross-linking with a user 20, or also any other protagonists of the healthcare sector. In this respect, the server 1 forms the central element of the computer system. The server 1 is equipped with various different communications interfaces 30 to 36. Communication with the user 20 is enabled via the interface 30. The user 20 can, in that regard, use a special device for user communication 22, such as a mobile user terminal, for example, a smartphone or tablet. The user can use a measuring device 21 to obtain his/her objective health data. It is possible for this measuring device 21 to consist of a digital measuring device, likewise with a communications interface. This then allows for recording data obtained for the user with the measuring device directly onto the server 1 and the corresponding database 2 of the server 1. This is, however, not absolutely necessary. A user 20 can also obtain measured values concerning his/her state of health with conventional analog devices (such as conventional bathroom scales or a conventional blood pressure monitor).

In the example according to FIG. 6, a doctor 23, a pharmacy 24, a health insurance company 25, a specialist doctor 26, and a hospital 27 are communicatively linked to the system 1 as further protagonists of the healthcare sector. Depending upon the function of the protagonist, the protagonist is linked to a specific communications interface 30 to 36. It is, for example, the case that all registered community GPs 33 can interact with the server via the same communications interface 31 or the same type of communications interface 31. Particular access rights to the server 1 or particular data privacy provisions are implemented via the user of a certain communications interface 30 to 36. Health data in the system in accordance with the present invention can thereby be comprehensively exchanged between various different protagonists, for example, in real time and in a fully automated way.

The method, computer program product and system in accordance with the present invention offer great opportunities to sustainably improve the healthcare landscape. By implementing special communications technology, it can be achieved that health data is exchanged, evaluated and used to benefit the patient to the fullest extent possible.

The present invention is not limited to embodiments described herein; reference should be had to the appended claims.

What is claimed is:

1-20. (canceled)

21. A method for exchanging health data between a computer system and a user, the method comprising:
providing a user database comprising contact details and health data of the user, the user database being structured so that the user is allocated to a user class based on the health data;
providing a medical database comprising entries relating to user class-specific health questions, a number of predefined multiple choice answers for each of the user class-specific health questions, and user class-specific health recommendations;
providing a first communications interface in the computer system for the user of the computer system;
selecting, via the computer system, a user class-specific health question from the medical database for the user,
selecting, via the computer system, a user class-specific health recommendation from the medical database for the user;
transmitting to the user the user class-specific health question selected from the computer system together with the predetermined multiple choice answer related thereto, wherein the computer system establishes a point in time when the user class-specific health question is to be transmitted based on details provided by the user in regard to the user's daily schedule and customs;
receiving, by the computer system, an answer selected by the user;
storing a state of health of the user in the user database based on the health data received in the form of the answer to the user class-specific health question;
transmitting the user class-specific health recommendation selected by the computer system from the computer system to the user;
once a predetermined recommendation time interval has lapsed, transmitting a query to the user concerning whether the user class-specific health recommendation sent to the user from the computer system has been implemented;
receiving an answer of the user regarding whether the user has implemented the user class-specific health recommendation; and
saving in the user database a conduct of the user in regard to whether the user has implemented the user class-specific health recommendation.

22. The method as recited in claim 21, wherein the user class is defined based on at least one of an age of the user, a sex of the user, a lifestyle of the user, an illness, a degree of severity of the illness, a complication associated with the illness, and a medication.

23. The method as recited in claim 22, wherein the user class is further defined by a combination of a main class and at least one sub-class.

24. The method as recited in claim 22, further comprising:
regularly checking an allocation of the user to the user class and, if necessary,
updating the user class of the user accordingly.

25. The method as recited in claim 21, wherein a data set for the user class-specific health question comprises a content parameter which permits an individualized linking with the health data of the user.

26. The method as recited in claim 21, wherein at least one user class-specific health question in the medical database is allocated a time interval for repetition as a parameter which specifies at what time interval a same user class-specific health question is re-sent to the user.

27. The method as recited in claim 21, wherein the health data stored in the medical database comprises both objective health data and subjective health data.

28. The method as recited in claim 27, wherein the health data stored in the medical database further comprises at least one health parameter selected from blood pressure, pulse, body weight, body fat percentage, and blood sugar.

29. The method as recited in claim 21, wherein,
one or more steps are executed repeatedly, in whole or in part, and
at least one of the user class-specific health question and the user class-specific health recommendation is selected by the computer system based on a feedback loop.

30. The method as recited in claim 21, further comprising:
evaluating the user in regard to a usage pattern of the user in regard to the computer system, and in regard to an execution by the user of the user class-specific health recommendations.

31. The method as recited in claim 21, further comprising:
providing a second communications interface for a healthcare sector participant, the healthcare participant comprising a doctor, a specialist doctor, a hospital, a pharmacy, and a health insurance company.

32. The method as recited in claim 31, further comprising:
automatically exchanging health data relating to the user between the computer system and the healthcare sector participant.

33. The method as recited in claim 32, further comprising:
a plurality of healthcare sector participants,
wherein,
the health data relating to the user is automatically exchanged between the computer system and the plurality of healthcare sector participants in at least one of at once and in real time.

34. The method as recited in claim 21, further comprising:
analyzing at least one of an efficacy and a cost-effectiveness of a health-promoting measure.

35. A computer program product comprising a program code for executing the method as recited in claim 21 in the computer system.

36. A system for exchanging health data between a server and a user to perform the method as recited in claim 21, the system comprising:
a user database comprising contact details and health data of the user, the user database being structured so that the user is allocated to a user class based on the health data;
a medical database comprising user class-specific health questions, a number of possible multiple choice answers for each of the user class-specific health questions, and user class-specific health recommendations;
a first program code programmed to manage a communication between the server and the user;
a second program code for automatic healthcare assistance, the second program code being programed,
to automatically select a user class-specific health question with a possible multiple choice answer associated therewith to be sent to the user, and
to select a user class-specific health recommendation for the user;
a third program code programmed to automatically record a state of health of the user based on the health data transmitted to the server by the user in the form of an answer; and
a fourth program code programmed to automatically control an execution of the user-specific health recommendation received by the user based on details transmitted by the user to the server,
wherein,
the system establishes a point in time for the transmission based on details given by the user in regard to the user's daily schedule and customs.

37. The system as recited in claim 36, further comprising:
a second communications interface for a healthcare sector participant, the healthcare participant comprising a doctor, a specialist doctor, a hospital, a pharmacy, and a health insurance company.

38. The system as recited in claim 37, further comprising:
a fifth program code programed for an automatic communication between the server and the healthcare sector participant,
wherein,
the automatic communication exchanges the health data of the user between the server and the healthcare sector participant.

39. The system as recited in claim 38, wherein the health data relating to the user is automatically exchanged between the server and a plurality of healthcare sector participants in at least one of at once and in real time.

40. The system as recited in claim 36, further comprising:
a mobile user terminal selected from at least one of a smartphone and a tablet, the mobile user terminal being configured so that the user can communicate with the system.
